(19) United States
(12) Patent Application Publication
(10) Pub. No.: US 2021/0309957 A1
(43) Pub. Date: Oct. 7, 2021

(54) APPARATUS FOR MONITORING GAS MOLECULES IN FERMENTATION BASED PROCESSES

(71) Applicant: VAYU SENSE AG, Munich (DE)

(72) Inventors: Amir BIRO, Tzur Moshe (IL); Moria SHIMONI, Petach Tikva (IL)

(73) Assignee: VAYU SENSE AG, Munich (DE)

(21) Appl. No.: 17/057,265

(22) PCT Filed: Jul. 7, 2019
(86) PCT No.: PCT/IL2019/050750

§ 371 Date: Nov. 20, 2020

(30) Foreign Application Priority Data
Jul. 10, 2018 (IL) 260523

(51) Int. Cl. C12M 1/34; C12M 1/36; G01N 21/3504; G01N 21/39; G01N 33/497

(52) U.S. Cl. CPC C12M 41/34; C12M 41/48; G01N 2021/396; G01N 21/39; G01N 33/497; G01N 21/3504

(57) ABSTRACT

An apparatus configured to perform in-situ, real-time, noninvasive monitoring of fermentation processes at a location remote from the fermenter/bioreactor in a laboratory or an industrial environment is described. The apparatus enables monitoring and detection of CO₂ in fermentation-based processes with high precision, continuously and in real time from the exhaust pipe or exhaust bypass of any size or type of fermenter/bioreactor or pipe diameter.

### CROSS-REFERENCE TO RELATED APPLICATIONS

This application is a continuation of International Patent Application No. PCT/IL2019/050750 filed on Jul. 7, 2019, now pending, which claims the benefit of Israel Application No. 260523 filed on Jul. 10, 2018. The contents of the above-referenced applications are hereby incorporated by reference.

### FIELD OF THE INVENTION

The invention is from the field of monitoring biological processes. Specifically, the invention is from the field of in-situ, real-time, noninvasive gas monitoring of fermentation processes.

### BACKGROUND OF THE INVENTION

In fermentation processes specific microorganism species are deliberately introduced into a fermentation container containing a material serving as growth medium in a metabolic process that consumes carbon source and other nutrients. The fermentation container is kept at suitable conditions (e.g. pH, agitation, temperature, etc.) and suitable growth medium encouraging growth of microorganisms and/or production of desired product by microorganisms using different processes.

For example, fermentation processes are used in the biotech industry for generating various biological cells such as: microbial cells (such as E. coli); fungi cells, yeast cells, and biological substances such as enzymes (catalase, amylase, protease, etc.); primary metabolites (ethanol, citric acid, glutamic acid, etc.); recombinant proteins and secondary metabolites (antibiotic, recombinant products: insulin, hepatitis B vaccine, interferon, etc.). In the food and beverage industries during conversion of carbohydrates into alcoholic beverages cells produce CO₂ via metabolic cycles while cells grow and proliferate.

Current methods applied today (using techniques such as Optical Density, Live Counts, or glucose concentration/consumption) for tracking cells or microorganism growth and determining the optimal growth conditions need invasive sampling and therefore are prone to errors. Other online methods, such as pH or dO₂ measurements are not considered to be accurate to correlate with biomass. A requirement of industry is an online, high resolution measurement system that enables, monitoring, controlling and optimizing in real-time, fermentation-based production processes in all scale fermenters. Among the processes that this solutions applies to (but not exclusively) are the production of small molecules, bio-similars, APIs, recombinant proteins, and vaccines.

High accuracy CO₂ measurements are important in terms of predicting process stage and trend. CO₂ value is a significant parameter in the growth, secondary metabolites biosynthesis and maintenance. During fermentation processes better CO₂ monitoring and precise detection capabilities are essential for analyzing the culture nonlinear dynamics and multistage nature that can be reflected with high correlation by CO₂ measurements.

Typically, a gas inlet is coupled to a fermentation container to supply suitable conditions for the microorganisms' metabolism and a gas outlet is also coupled to the fermentation container to remove waste gases, throughout an exhaust pipe.

In many cases, monitoring of fermentation processes is done by a combination of on-line and occasional offline sampling. The material in the fermenter/bioreactor is sampled and analyzed, in order to determine parameters reflecting the concentration of the microorganisms/cells and/or the yield/titer of the product in the process, thereafter, utilizing that data to control the fermentation process.

Conventional techniques for monitoring of some of the fermentation parameters are not performed continuously and may not be carried out with high accuracy in real-time. Typically, these measurements involve sporadic sampling of the fermenter/bioreactor and analysis methods that require the know-how and accuracy of a technician. Amongst these techniques are optical/critical density measurement, viable counts, and metabolites consumption in the growth media by measuring yield/titer of the product. Using these methods, the optimal time points for process harvest are often undetected. Other continuously methods such as pH, dO₂ etc. are crucial for process control but cannot reflect biomass.

Co-pending patent application US 2017/0267964 to the applicant of the present application contains a description of a system and method that provides accurate real time and continuous/planned monitoring of fermentation processes by sampling the gas emission in a continuous/programmed manner. According to the rate and composition of the metabolic gas emitted from the fermenter/bioreactor. The collected data is based inter alia on the rate of metabolic gas production by microorganisms/cells contained therein or a change in such rate of production. From this data is determine the amount of the microorganisms/cells, biomass, or the rate of change in their growth rate.

The method described in US 2017/0267964 is based on measuring the absorption of illuminating light (typically in the infrared spectrum) transmitted through a gaseous atmosphere in fluid communication with the fermentation material, i.e. in the dead space above the fermentation material. Living microorganisms produce metabolic gases such as carbon dioxide (CO₂) during respiration. By means of infrared absorption, the concentration of metabolic gases given off by the fermentation process can be measured inside the dead space.

FIG. 1 schematically illustrates in a block diagram a system 10 according to some embodiments disclosed in US 2017/0267964 for detection of metabolic gases in a fermentation process. The system 10 includes an optical system which includes a tunable broadband IR light source 12 and a detection module 15. The tunable broadband IR light source 12 is configured and operable for emitting light in a predetermined substantially narrow spectrum. The tunable broadband IR light source 12 is controllably operated for emitting light in at least a first and a second predetermined wavelengths, wherein the first wavelength corresponds to an absorption peak of at least one metabolic gas, typically CO₂, to be detected, and the second predetermined wavelength is in a spectral region outside the absorption peak of the at least one metabolic gas. The detection module 15 includes a detector 14 sensitive in the IR wavelength regime and signal processing components 13. The light source 12 and the detection module 15 are aligned to form illumination and detection paths respectively, e.g. being in optical communication with one another, intersecting a region of interest. The detection module is configured and operable for detecting light in the first and second wavelengths, such as light 20 passing through the region of interest located in between the light source 12 and the detector 14, and generating intensity data/signals indicative of the intensity of detected light in the at least first and second wavelengths. This data is therefore indicative of the transmittance of the region of interest to the at least said first and second wavelengths.

Further provided in the system 10 is a control system 30 (e.g. controller), which is connectable to the optical system, i.e. to the light source 12 and to the detection module 15. The controller 30 is configured and operable for operating the light source 12 to emit light in the selected at least first and second wavelengths, and for receiving and analyzing measured/detected data/signals from the detection module and generating data indicative of a concentration of the metabolic gas in the region of interest.

In some embodiments, the light source 12 and the detector 14 are arranged in spaced-apart relationship defining the region of interest between them for spectroscopic measurements. To this end, light source 12 and the detector 14 are arranged such that a suitable container 24 of a fermenting material 26 and/or more specifically a dead space 28 associated with and being in fluid communication with such container 24 can be placed. The dead space 28 is where metabolic gases should be detected by optical/spectroscopic measurements performed by the system 10 of the present invention. As indicated above, the dead space 28 of the container is actually any space being in fluid communication with the atmosphere in the container above fermenting material 26. This may include any one of the following: the portion 28 of the container 24 above the fermenting material as illustrated for example in FIG. 1, and/or any suitable closed gas-chamber such as a reservoir and/or an outlet pipe/tube connected to the container and being in fluid communication with the atmosphere inside the fermentation container 24.

The analysis of the measured data is generally based on the principles of spectroscopy. The first and second wavelengths are particularly selected to enable accurate and high-sensitivity measurements of the concentrations of metabolic gas, with absorption specific only to the desired gas molecule being measured with no cross-interference by any other molecule in the measurement volume. The first wavelength is selected to be highly affected by absorbance by the at least one metabolic gas in the region of interest, i.e. to overlap with the spectral absorption line. The second wavelength is on the other hand selected to be less affected by absorbance of the metabolic gas, but nevertheless it is selected to be spectrally close to the first wavelength, such that it provides reference data indicative of absorbance of the first wavelength by other materials in the region of interest.

In US 2017/0267964 there is described in detail the method of obtaining the data and analyzing it and also the required properties of the various components of the system. However, the system is limited to making measurements inside the fermentation container. US 2017/0267964 supplies no description of a specific practical system that could be connected to a fermentation container in order to make in-line measurements at a location remote from the container in which the fermentation process is taking place in a laboratory or an industrial environment.

It is therefore a purpose of the present invention to provide an apparatus configured to perform in-situ, real-time, noninvasive monitoring of fermentation processes at a location remote from the fermenter/bioreactor in a laboratory or an industrial environment.

It is another purpose of the invention to provide an apparatus configured to perform online, high resolution measurements that enable monitoring, controlling, and optimizing in real-time fermentation-based production processes in all scale or type of fermenters.

Further purposes and advantages of this invention will appear as the description proceeds.

### SUMMARY OF THE INVENTION

An apparatus configured to perform in-situ, real-time, noninvasive monitoring of fermentation processes at a location remote from the fermenter/bioreactor is described. The apparatus comprises:
a) a light source and detection module (LSDM);
b) an exhaust tube adapter (ETA), which is coupled to or inserted into an exhaust tube of the fermenter/bioreactor and bolted onto a front face of the LSDM; and
c) a control and display module, which is in communication with the LSDM via a wired or wireless communication channel.

In embodiments of the apparatus the LSDM comprises:
a) a light source;
b) a detector;
c) electrical components necessary to operate the light source and at least some of the components necessary to process output signals from the detector; and
d) optical components to direct a light beam from the light source out of the LDSM into the ETA and a light beam returning from the ETA into the LDSM onto the detector.

In embodiments of the apparatus the light source is a broadly tunable light source having a tunable range of at least 2 cm⁻¹ and emissions in the spectral range of the metabolic gas being observed.

In embodiments of the apparatus the metabolic gas is CO₂ and the spectral range is 2100-2400 cm⁻¹.

In embodiments of the apparatus the light source is a tunable Quantum Cascade Laser.

In embodiments of the apparatus the ETA is connected to the exhaust tube or a bypass to the exhaust tube by two pieces of flexible tubing, wherein a first piece of flexible tubing leads metabolic gas from the fermenter/bioreactor into the ETA and a second piece of flexible tubing leads metabolic gas out of the ETA.

In embodiments of the apparatus the ETA comprises:
a) a casing;
b) a flange configured to be bolted to a matching flange or threaded holes on the LSDM; and
c) two rigid tubes that are in gas communication with each other and are hermetically closed with windows at their ends closest the LSDM and, at their other ends, by an arrangement comprised of a mutual window with a mirror behind it or by a mirror;
wherein the two rigid tubes and the mirror are arranged such that a light beam exiting the LSDM passes through one of the rigid tubes, is reflected from the mirror, and returned through the second rigid tube into the LSDM; and
a first piece of flexible tubing connects between the exhaust tube of the fermenter/bioreactor to an inlet to one of the rigid tubes in order to allow metabolic gas to enter the two rigid tubes and a second piece of flexible tubing connects between an outlet of the second rigid tube and the exhaust tube of the fermenter/bioreactor in order to allow metabolic gas to exit the two rigid tubes.

In these embodiments of the apparatus the ETA can be disconnected from the LSDM and can be discarded after a fermentation process.

In embodiments of the apparatus the ETA comprises:
a) an interface configured to connect the ETA to a LSDM;
b) a housing; and
c) a multi-pass system for a light beam that is directed into the ETA from the light source in the LSDM and exits the ETA to be directed onto the detector in the LSDM.

In these embodiments of the apparatus two opposing sides of the housing of the ETA comprise holes that are hermetically connected to the respective ends of a gap in the exhaust tube of the fermenter/bioreactor, the size of the housing depends on the diameter of the exhaust tube to which the ETA is connected, and the interface has the same dimensions for all sizes of housing.

In embodiments of the apparatus comprising a multi-pass system the multi-pass system is configured such that the number of passes of the light beam is dependent on the size of the housing to achieve the same optical path for housings of all sizes.

In embodiments of the apparatus the control and display module comprises:
a) a processor;
b) dedicated software configured to operate the light source in the LSDM, to receive data from the LSDM, to analyze the data, and output information relating to the status of the fermentation process;
c) data bases to store historical data; and
d) input, output and display means, wherein the input, output and display means comprise at least one of a keyboard, computer monitor, printer, and touch screen graphical user interface.

All the above and other characteristics and advantages of the invention will be further understood through the following illustrative and non-limitative description of embodiments thereof, with reference to the appended drawings.

### BRIEF DESCRIPTION OF THE DRAWINGS

FIG. 1 schematically illustrates in a block diagram a prior art system for detection of metabolic gases in a fermentation process;

FIG. 2 schematically illustrates in a block diagram the system of the invention for detection, at the exhaust of a fermentation vessel, of metabolic gases formed during a fermentation process;

FIG. 3 is an external view showing ETA and an embodiment of a LSDM bolted together;

FIG. 4A is an oblique top view of the LSDM with the top of the casing removed to reveal the inner components;

FIG. 4B is an enlarged view of elliptical area A in FIG. 4A showing in more detail the optical components of the LSDM;

FIG. 5 shows a first embodiment of an ETA with the top of its casing removed to reveal the inner components;

FIG. 6 shows the ETA and LSDM bolted together;

FIG. 7 and FIG. 8 schematically show an embodiment of ETA for use with fermentation vessels having different diameter exhaust tubes; and

FIG. 9 schematically illustrates the path of the laser beam in the interior of the housing of the ETA shown in FIGS. 7 and 8.

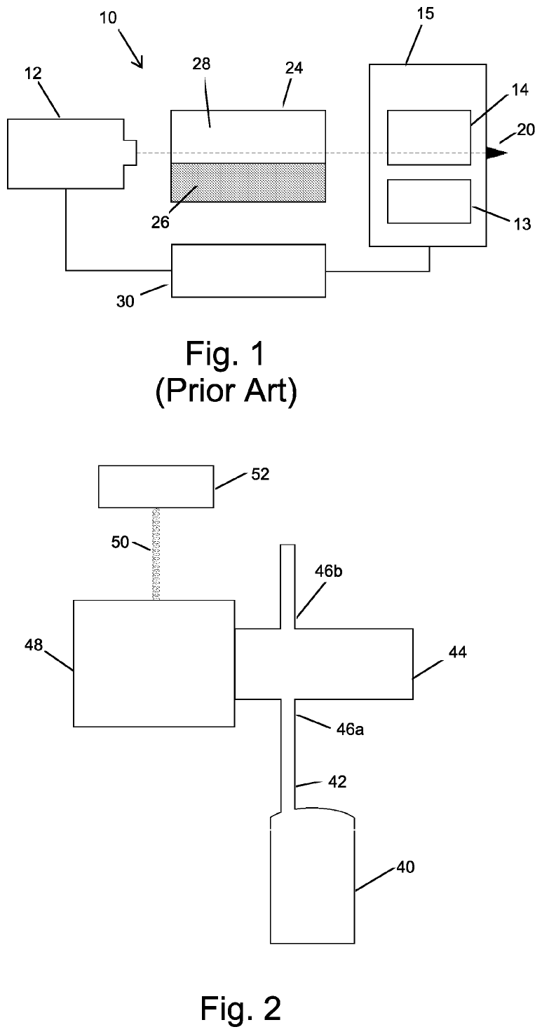

### DETAILED DESCRIPTION OF EMBODIMENTS OF THE INVENTION

Herein is described an apparatus configured to perform in-situ, real-time, noninvasive monitoring of fermentation processes at a location remote from the fermenter/bioreactor in a laboratory or an industrial environment.

The apparatus enables monitoring and detection of CO₂ in fermentation-based processes with high precision, continuously and in real time from the exhaust pipe or exhaust bypass of any size fermenter/bioreactor or pipe diameter.

The apparatus is comprised of a very sensitive, non-invasive meter device, called herein a light source and detection module (LSDM) that is removably attached to an exhaust tube adapter (ETA) or to a disposable bypass from fermenter exhaust pipe and a control and display module.

The ETA, assembled in-line with the fermentation vessel exhaust pipe, comes in different embodiments that enable the LSDM to provide continuous metabolic gas detection for highly accurate monitoring of the process in any size fermenter/bioreactor with same optical path.

The LSDM records and analyzes metabolic gas concentrations, CO₂ produced during the respiration, and growth of living cells. Continuous, automatic measurements via an IR optical system allows the in-situ detection of metabolic gases without interrupting the process for invasive sampling.

FIG. 2 schematically illustrates in a block diagram the system of the invention for detection of metabolic gases in a fermentation process carried out in fermentation vessel 40. The system is comprised of three modules that are connected to the output tube of fermentation vessel 40. Light Source and Detector Module (LSDM) 48 comprises, as its name implies, a light source, detector, at least some of the electronic components necessary to operate the light source and detector, optical components to direct a beam of light from the light source out of the LDSM into the Exhaust Tube Adaptor (ETA) 44 and the beam of light returning from the ETA into the LDSM onto the detector, and at least some of the components necessary to process the output signals from the detector. Exhaust Tube Adaptor (ETA) 44 is inserted into the exhaust tube 42 of the fermentation vessel 40 and is bolted onto the front face of LSDM 48. Metabolic gases produced during a fermentation process that takes place in fermentation vessel 40 exit the fermentation vessel through exhaust tube 42 and flow through the ETA 44 entering ETA 44 at inlet 46a and exiting at outlet 46b. Two embodiments of ETA will be described herein—the first for relatively small diameter exhaust tube or bypass to the exhaust tubes 42 and the second for variable diameter exhaust tubes 42. The ETA 44 can be provided to be coupled into exhaust tubes having a wide range of diameters, e.g. from 1″ to 8″ in 0.25″ steps, although in practice there is no upper limit to the diameter.

The third module of the system is control and display module 52, which is in communication with the LSDM 48 via a wired or wireless communication channel symbolically shown by cable 50. The functions of the components of control and display module 52 can be provided by any combination of components or devices that include a processor, communication functions, and a graphic user interface. One example of a control and display module 52 that is suitable for use with the system is a personal computer that comprises dedicated software configured to operate and receive data from the LSDM 48, to analyze the data, and output information relating to the status of the fermentation process.

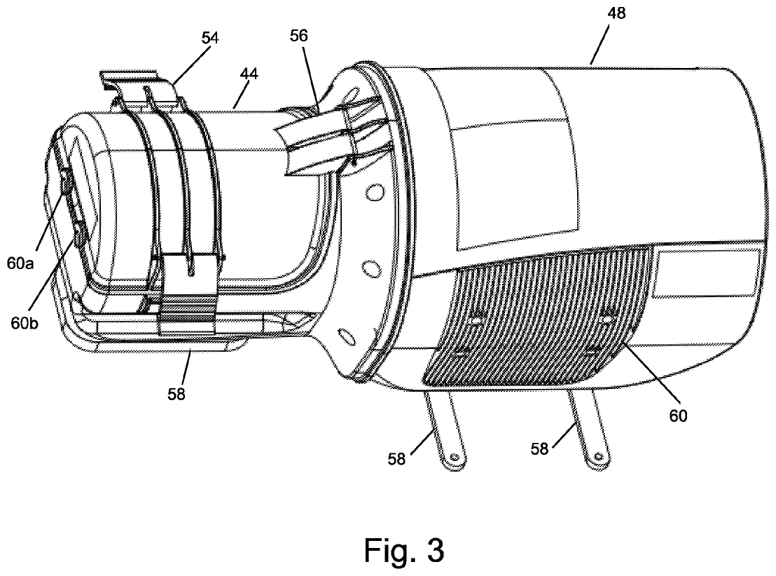

FIG. 3 is an external view showing ETA 44 and the first embodiment of a LSDM 48 bolted together. Seen on the front face of the face of ETA 44 are ports 60a and 60b through which hoses or tubes pass to be connected respectively to inlet 46a and outlet 46b (see FIG. 2) as will be described herein below. In the embodiment shown, quick release clasps 54 allow the top part of the housing of the ETA to pivot on hinges 56 to allow easy access to the interior of ETA 44 for connection to inlet 46a and if necessary outlet 46b (In some scenarios the gases exiting the fermentation vessel 40 may be exhausted to the atmosphere after passing through ETA 44). In other embodiments the top and bottom parts of the housing may be bolted together.

Mounts 58 are provided to support LSDM 48 and ETA 44. Grill 60 on the side of the casing of LSDM 48 is provided to allow heat generated by the light source and electronic components within the casing to be expelled to the surroundings.

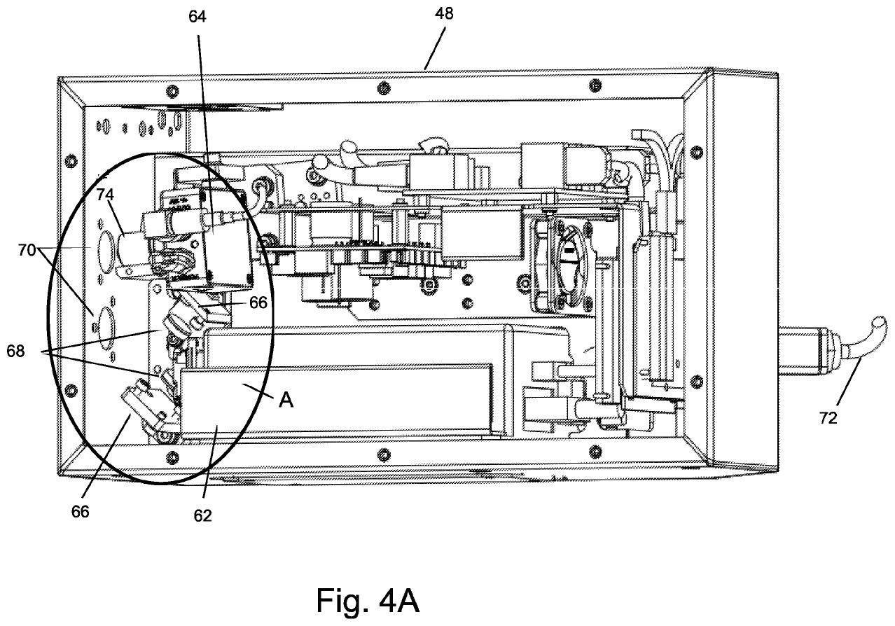

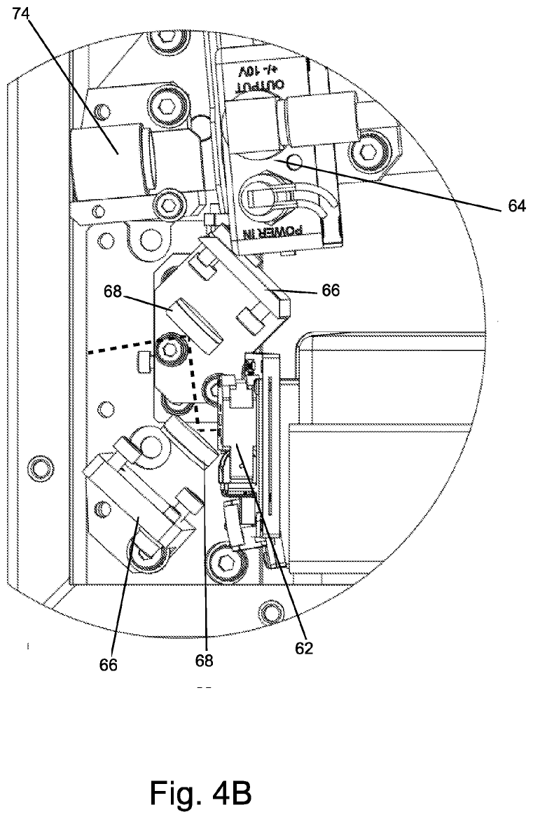

FIG. 4A is an oblique top view of LSDM 48 with the top of the casing removed to reveal the inner components. The electronic circuitry and other components not essential to understanding the invention are not described herein.

FIG. 4B is an enlarged view showing in more detail the optical components in elliptical area A in FIG. 4A. Seen in both figures are laser 62, mirror mounts 66, two folding mirrors 68 and focusing optics 74 that focus the beam returning from the ETA 44 onto detector 64. In FIG. 4A are seen power cord 72 and two windows 70 on the front wall of the casing, which are transparent to the wavelength used, through which the laser beam can exit and reenter the LSDM 48 to and from the ETA 44. Embodiments of the LSDM can comprise one large opening instead of the two shown in FIG. 4A. The windows 70 can have different shapes, e.g. elliptical or rectangular. In FIG. 4B the dashed line shows the path of the laser beam from the laser to the opening 70 on its way to the ETA 44.

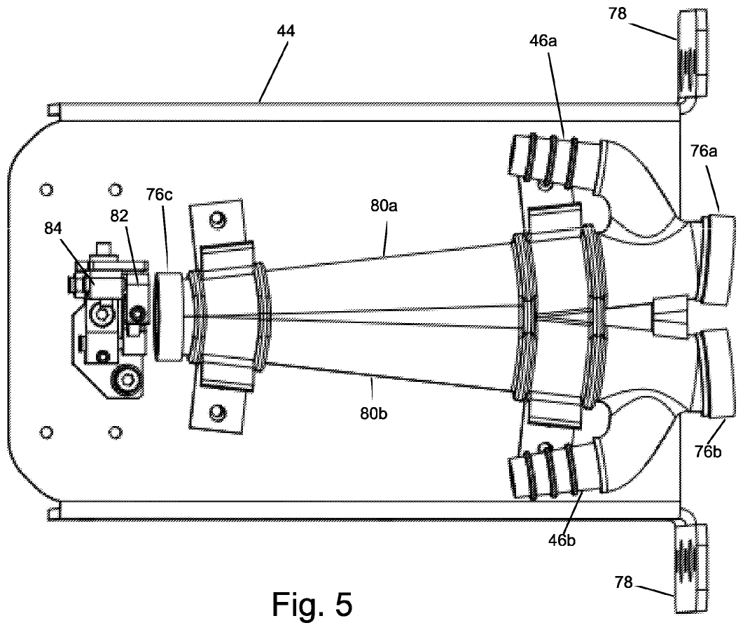

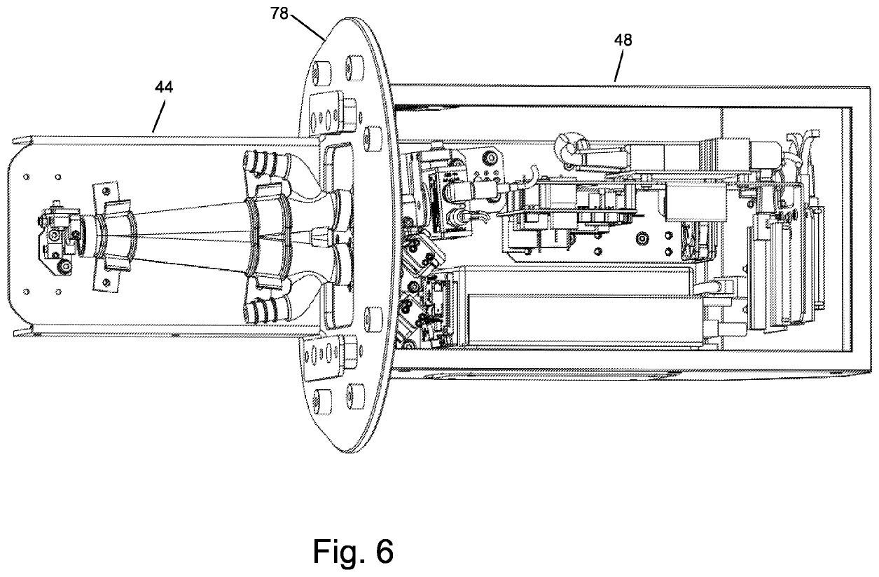

FIG. 5 shows a first embodiment of an ETA 44 with the top of its casing removed to reveal the inner components. A flange 78 is configured to be bolted to a matching flange or threaded holes on the LSDM 48 as shown in FIG. 6. In order to provide a sufficiently long optical path to allow an accurate measurement of the concentration of the metabolic gas, ETA 44 comprises two rigid tubes 80a and 80b that are in fluid communication with each other. Each of the two tubes 80a and 80b are hermetically closed at the end closest the LSDM by windows 76a and 76b respectively and another window 76c hermetically seals the ends of both tubes at their other ends. Gaseous effluent containing the metabolic gas whose concentration is to be measured flows from the fermentation vessel 40 to ETA 44 and enters tube 80a through inlet 46a, flows to the end of tube 80a, wherein a mutual opening with tube 80b allows the gas to flow through tube 80b to outlet 46b, and is discharged back to the exhaust tube 42 of the fermentation vessel 40 or to the atmosphere if local environmental regulations permit. For this embodiment of ETA 44, the connections between the exhaust tube 42 of the fermentation vessel 40 to inlet 46a and from outlet 46b back to the exhaust tube 42 (or the atmosphere) are made by means of flexible tubing (not shown in the figures) that enters the interior of ETA 44 through appropriately sized openings 60a and 60b (see FIG. 3) in a wall of the ETA.

Windows 76a and 76b are facing windows 70 in the wall of LSDM 48 when ETA 44 and LSDM 48 are bolted together as shown in FIG. 6. Located behind window 76c in ETA 44 is folding mirror 82 in mirror mount 84. Light from the laser 62 is reflected by mirrors 66, which direct it through window 76b. The laser beam then travels through tube 80b until it reaches mirror 82, which redirects if back through tube 80a and out of window 76a to lens 74 and detector 64. In other embodiments of the ETA window 76c can be a mirror, which eliminates the need for mirror 82 and mirror mount 84.

In order to keep air paths to a minimum length to minimize absorption by CO₂ and other atmospheric gases, cylindrical spacers made of an infrared transmitting material, e.g. sapphire, are placed along the optic axis between the optical components to physically reduce the air path.

The method of determining the progress of a fermentation process is based on monitoring the concentration of a metabolic gas in the ETA, typically CO₂, by optical/spectroscopic measurements. The method, which is described in detail in U.S. Pat. No. 9,441,260 (including the equations used by the software in the processor to calculate the concentration of the metabolic gas of interest), comprises measuring at least a first and a second predetermined wavelength of substantially narrow spectrum corresponding to respectively an absorption peak of at least one metabolic gas and a spectral region outside the absorption peak of the at least one metabolic gas, and measuring transmission of the first and second wavelengths through the ETA. The spectral separation between the first and second wavelengths of the light source is selected to be small such that the first and second wavelengths are characterized by same or similar transmission through materials in the optical path. This allows the transmission measurements at the second wavelength to be used to correct the measurements at the first wavelength for absorption by the windows and mirrors in the ETA, components of the volatile gas emitted in the fermentation process other than the specific metabolic gas whose concentration is being measured, and air paths, which are kept to a minimum.

In order to obtain the two wavelengths required for determining the concentration of the metabolic gas, the light source must be a broadly tunable light source having a tunable range of at least 2 cm⁻¹ and emissions in the spectral range of the metabolic gas being observed, for example in the 2100-2400 cm⁻¹ range (about 4.3 microns) which corresponds to a spectral regime of high absorbance by CO₂. An embodiment of a light source that meets these requirements and is used in the system of this invention is a tunable Quantum Cascade Laser (QCL) 62. QCL 62 is used as the light source, since, in addition to being tunable over a wide wavelength range, a QCL also provides sufficiently narrow spectral width, i.e. sufficiently monochromatic light emission. Another possible light source that can be used is a broadband source equipped with suitable narrow-band spectral filters in the mid-IR regime.

In a typical embodiment of the system for measuring the concentration of CO₂, the system utilizes a tunable QCL 62, IR detector 64, a CaF₂ plano-convex lens 74, front surface coated mirrors 68,82, and sapphire windows 70 and 76a,b,c. The QCL operates, for example, in the pulse mode with repetition frequency of 5 kHz and pulse width 500 nsec. It is easily within the ability of persons skilled in the art to replace certain of these components and materials mutatis mutandis with similar components and materials that are suitable for detecting metabolic gases other than CO₂ and/or in other spectral regions.

Embodiments of the LSDM 48 include an electronic signal processor/lock-in amplifier (whose components are shown but not labelled in the figures) that receives signals from the IR detector 64 and a control and display module 52. Use of the lock-in amplifier enables even further improvement of the signal to noise ratio (SNR) provided by the system thus further improving the sensitivity and accuracy of the measurements relating to the concentration(s) of metabolic gases and consequently to control of the fermentation process. To this end, in such embodiments the control system 52 is adapted for operating the tunable broadband IR light source 62 for applying time modulation to intensity of light emitted in one or more (e.g. in each) of at least two (first and second) wavelengths, and also operating the lock-in amplifier to determine/measure the detected intensity/intensities of the emitted light with high accuracy based on that modulation. Accordingly, transmittance of the region of interest to the first and second wavelengths (i. e. to all wavelengths used in the measurement) can be determined with high accuracy based on the intensity modulation, while noise is mostly discarded as it is generally not modulated in the same way. It should be noted that the configuration and operation of various types of lock-in amplifier are generally known in the art of signal processing and are therefore not specifically described herein. A person versed in this art would readily appreciate the various possible configurations of such lock-in amplifier with appropriate modulation to the emitted illumination to be used in the system of the invention.

The control and display module 52 is configured for operating the broadly tunable IR light source for modulating light intensity of the at least first and second wavelengths, and operating the lock-in amplifier or an Excitation/Oscillation clock to determine the transmission of the region of interest to the at least first and second wavelengths with high signal to noise ratio based on the modulation. The control and display module 52 also comprises software containing equations and algorithms for transforming the raw data from the detector into concentration of the metabolic gas of interest, data bases to store historical data, and input, output and display means, e.g. a keyboard, computer monitor, printer, and touch screen GUI.

The embodiment of the ETA described herein above is designed for use with fermentation vessels having different diameter exhaust tubes. When used, for example, with a pathogenic culture, this embodiment of ETA can be disconnected from the LSDM and discarded after each fermentation process saving a time-consuming cleaning and sterilization process. The LSDM is never in contact with the gases exiting the fermentation vessel and therefore requires no cleaning and can be connected to another ETA to monitor another process taking place in the same or a different fermentation vessel.

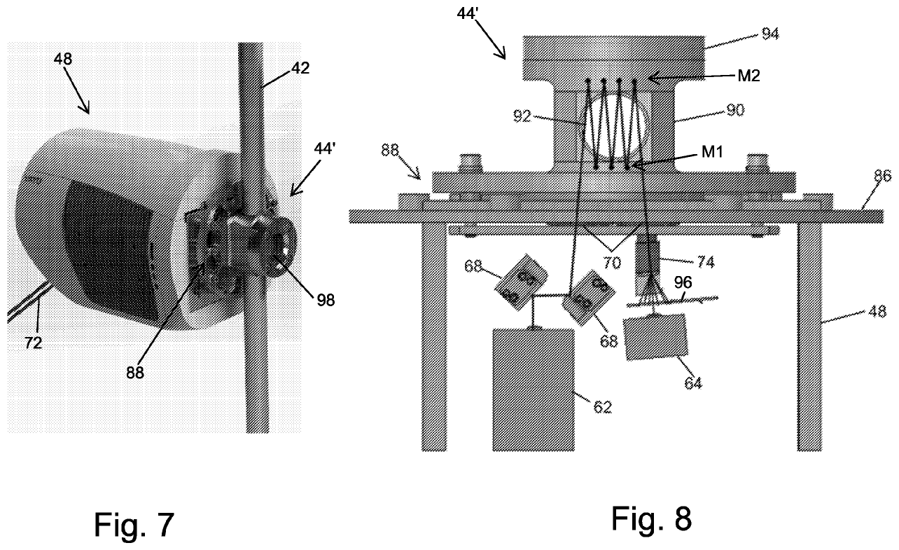

FIG. 7 is a photograph showing ETA 44′ connected to the front of LSDM 48 by an interface 88. The LDSM 48 is identical to that described herein above. At the opposite end of ETA 44′ is seen a recess 98 into which a mirror of a multi-pass optical system described herein below is housed.

This embodiment of ETA is installed into a gap in the exhaust tube 42 that carries the metabolic gas away from fermentation vessel 40. Two opposite sides of the housing 90 contain holes that allow the interior of ETA 44′ to be filled with metabolic gas that exits the fermentation vessel through exhaust tube 42. The housing 90 of ETA 44′ is hermetically connected to the respective ends of exhaust tube 42 either by welding as shown in FIG. 7 or by means of flanges that are bolted for easy removal of ETA 44′ and connection to a different fermentation vessel. Between processes the ETA 44′ is not removed from the exhaust tube for sterilization.

FIG. 8 is a horizontal cross-section showing schematically only the critical components of the ETA 44′ and LSDM 48. The housing 90 of ETA 44′ is bolted to the front 86 of LSDM 48 via an interface 88. Interface 88 is designed such that is uses the same bolt holes in the front 86 of LSDM 48 that are used for connection of flange 78 of ETA 44. A beam of light from laser 62 is directed by two folding mirrors 68 and exits LSDM 48 through an IR transmitting window 70, e.g. made of sapphire or silicon for wavelengths within the mid-IR spectrum. From window 70, the light beam enters the interior of ETA 44′ through a window. In order to provide a sufficiently long optical path to allow an accurate measurement of the concentration of the metabolic gas, the interior of ETA 44′ comprises an arrangement of two mirrors M1 and M2 that forms a multi-pass system 92 that is shown schematically in the figure. After making a predetermined number of passes in the interior of ETA 44′, the light beam exits ETA 44′ through a window and enters LSDM 48 passing through focusing optics 74 and pinhole/slit 96 (to reduce power) to detector 64. M1 is conveniently provided by adding a reflective coating to a central portion of the interior surface of an IR transmitting window leaving a transparent border around the M1 through which the light beam can enter and exit the interior of ETA 44′. Cover Plate 94 seals the top of the housing 90 of ETA 44′ and holds M2 in place in recess 98 (see FIG. 7).

In order to connect to exhaust tubes 42 having larger diameters, the dimensions of housing 90 of ETA 44′ had to be increased/were increased. The dimensions of interface 88 are the same for all housing 90 sizes in order to be able to use the same LSDM 48 with fermentation vessels having exhaust tube with a large range of diameters.

As the dimensions of the housing 90 increase the number of passes of the laser light beam through the gas in the housing can be decreased. In many cases this can be accomplished without any changes to the optical system but in some cases, in order to keep the same optical path between where the beam enters and exits housing 90 of ETA 44′, the angle at which the beam enters must be slightly (typically by 1-3 degrees) altered by rotating the folding mirrors 68 in LSDM 48.

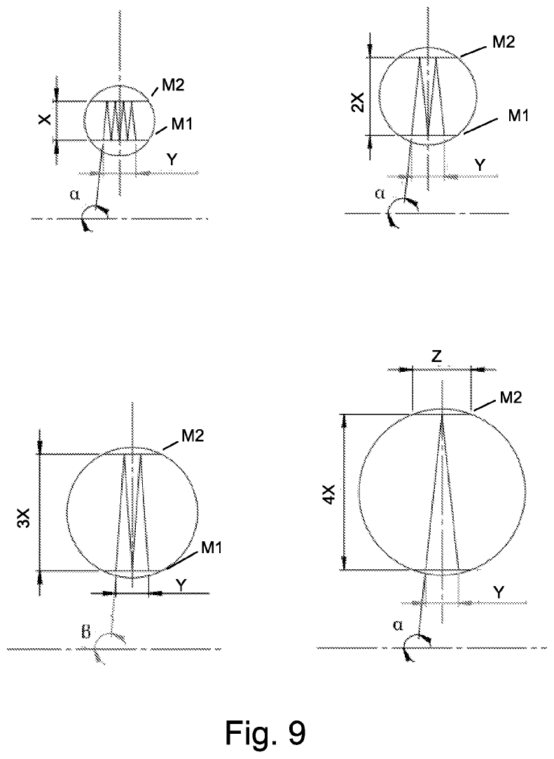

FIG. 9 schematically illustrates the path of the laser beam in the interior of the housing 90 of ETA 44′ for four different housing sizes. The interiors of ETAs 44′ comprise two mirrors M1 and M2 that form the multi-pass system 92 shown in FIG. 8. The number of passes and distance between M1 and M2 is determined such that the laser beam travels the same distance through the interior of the ETA, regardless of the diameter of the exhaust tube of the fermentation vessel to which the ETA is connected. In the examples shown in FIG. 9, for the smallest housing size the distance between mirrors M1 and M2 is X, which is the size shown in FIG. 8. Other sizes are 2X, 3X, and 4X. It is noted that for the ETA 44′ for which the distance is 4X mirror M1 is not needed since the beam is only required to make one round trip through the interior of ETA 44′. Y is the distance between entrance and exit windows of housing 90 and Z is the diameter of the cover plate 94. Both Y and Z are the same for all size housings. The entrance angles of the laser beam to the housing is designated by α and β. As shown for three of the sizes of the housing the entrance angle is the same, allowing for use of the same LSDM with any of these sizes.

Although embodiments of the invention have been described by way of illustration, it will be understood that the invention may be carried out with many variations, modifications, and adaptations, without exceeding the scope of the claims.

1. An apparatus configured to perform in-situ, real-time, noninvasive monitoring of fermentation processes at a location remote from a fermenter/bioreactor in which the process is taking place, the apparatus comprising:
a) a light source and detection module (LSDM);
b) an exhaust tube adapter (ETA), which is coupled to or inserted into an exhaust tube of the fermenter/bioreactor and bolted onto a front face of the LSDM; and
c) a control and display module, which is in communication with the LSDM via a wired or wireless communication channel.

2. The apparatus of claim 1, wherein the LSDM comprises:
a) a light source;
b) a detector;
c) electrical components necessary to operate the light source and at least some of the components necessary to process output signals from the detector; and
d) optical components to direct a light beam from the light source out of the LDSM into the ETA and a light beam returning from the ETA into the LDSM onto the detector.

3. The apparatus of claim 2, wherein the light source is a broadly tunable light source having a tunable range of at least 2 cm⁻¹ and emissions in the spectral range of the metabolic gas being observed.

4. The apparatus of claim 3, wherein the metabolic gas is CO₂ and the spectral range is 2100-2400 cm⁻¹.

5. The apparatus of claim 2, wherein the light source is a tunable Quantum Cascade Laser.

6. The apparatus of claim 1, wherein the ETA is connected to the exhaust tube or a bypass to the exhaust tube by two pieces of flexible tubing, wherein a first piece of flexible tubing leads metabolic gas from the fermenter/bioreactor into the ETA and a second piece of flexible tubing leads metabolic gas out of the ETA.

7. The apparatus of claim 6, wherein the ETA comprises:
a) a casing;
b) a flange configured to be bolted to a matching flange or threaded holes on the LSDM; and
c) two rigid tubes that are in gas communication with each other by and are hermetically closed with windows at their ends closest the LSDM and, at their other ends, by an arrangement comprised of a window with a mirror behind it or by a mirror;
wherein the two tubes and the mirror are arranged such that a light beam exiting the LSDM passes through one of the tubes, is reflected from the mirror, and returned through the second tube into the LSDM; and
first piece of flexible tubing connects between the exhaust tube of the fermenter/bioreactor to an inlet to one of the rigid tubes in order to allow metabolic gas to enter the two tubes and second piece of flexible tubing connects between an outlet of the second rigid tube and the exhaust tube of the fermenter/bioreactor in order to allow gas to exit the two rigid tubes.

8. The apparatus of claim 6, wherein the ETA is disconnected from the LSDM and discarded after each fermentation process.

9. The apparatus of claim 1, wherein the ETA comprises:
a) an interface configured to connect the ETA to a LSDM;
b) a housing; and
c) a multi-pass system for a light beam that is directed into the ETA from the light source in the LSDM and exits the ETA to be directed onto the detector in the LSDM.

10. The apparatus of claim 9, wherein two opposing sides of the housing of the ETA comprise holes that are hermetically connected to the respective ends of a gap in the exhaust tube from the fermenter/bioreactor.

11. The apparatus of claim 9, wherein the size of the housing of the ETA depends on the diameter of the exhaust tube to which the ETA is connected.

12. The apparatus of claim 11, wherein the interface has the same dimensions for housings of all sizes.

13. The apparatus of claim 11, wherein the multi-pass system is configured such that the number of passes of the light beam is dependent on the size of the housing to achieve the same optical path for housings of all sizes.

14. The apparatus of claim 1, wherein the control and display module comprises:
a) a processor;
b) dedicated software configured to operate the light source in the LSDM, to receive data from the LSDM, to analyze the data, and output information relating to the status of the fermentation process;
c) data bases to store historical data; and
d) input, output and display means, wherein the input, output and display means comprise at least one of a keyboard, computer monitor, printer, and touch screen graphical user interface.
